(19) United States
(12) Patent Application Publication
(10) Pub. No.: US 2018/0092772 A1
(43) Pub. Date: Apr. 5, 2018

(54) Automated Eye Drop Dispenser

(71) Applicant: Firas Emad Mosalam, San Diego, CA (US)

(72) Inventor: Firas Emad Mosalam, San Diego, CA (US)

(21) Appl. No.: 15/706,743

(22) Filed: Sep. 17, 2017

(30) Foreign Application Priority Data
Oct. 4, 2016 (IN) 201641034002

(51) Int. Cl. A61F 9/00

(52) U.S. Cl. CPC A61F 9/0026

(57) ABSTRACT

An apparatus for dispensing eyedrops comprising: a mechanism provided for controllably delivering a predetermined quantity of liquid; and an opaque eye cup provided for at least partially retracting the eyelids of a user's eye when applied around said user's eye; said eye cup having a distal aperture and being coupled to said mechanism such that said predetermined quantity of liquid drops through said distal aperture into a portion of the eye exposed by said at least partially retracted eyelids when said portion of the eye is maintained vertically aligned under said distal aperture.

### TECHNICAL FIELD

The present disclosure broadly falls in the technical field of eyedroppers and eye wash dispensers and more particularly is directed to an eye drop dispenser to be used in the self-administration of drops to the users' eyes.

### BACKGROUND

Human eye is extremely sensitive to dust and other foreign objects. A commonly accepted method of alleviating this condition is to apply a mild liquid medicant to the eyes, which is referred as an eyewash. At present, it is necessary to hold the head back and then drop the liquid into one's eyes from a dropper or dispenser bottle. This is done solely by the user by holding the dispenser in one's hand. This process has numerous disadvantages. One such disadvantage being that the liquid is lost without properly being placed, due to frequent blinking of the eye lid.

U.S. Pat. No. 5,607,410 discloses a fluid reservoir having a flexible squeeze container mounted on a housing. An eye piece at the other end of the trough is engaged which can be adjusted to the eye. U.S. Pat. No. 5,267,986 discloses a dispenser including a drop cavity which holds defined volume of fluid. A spring causes expansion of the dispenser to emit a drop through a nozzle. U.S. Pat. No. 5,336,190 provides a cassette assembly for an emergency medical infusion pump with a reservoir bag having a tube like container squeezable by a pump mechanism.

Known eye drop dispensers have not proven completely satisfactory, either because they are difficult to actuate or because they do not actually dispense reliably a predetermined quantity of liquid in the eye of a patient. There still exists a need for a dispenser system that allows dispensing effortlessly and reliably a predetermined quantity of liquid in the eye of a patient.

### SUMMARY

An embodiment of the present disclosure relates to a portable, compact, low-cost eye drop dispensing system using a pump that allows dispensing conveniently a predetermined quantity of ophthalmic liquid into a user's eye.

An embodiment of the present disclosure relates to an apparatus for dispensing eyedrops comprising: a mechanism provided for controllably delivering a predetermined quantity of liquid; and an opaque eye cup provided for at least partially retracting the eyelids of a user's eye when applied around said user's eye; said eye cup having a distal aperture and being coupled to said mechanism such that said predetermined quantity of liquid drops through said distal aperture into a portion of the eye exposed by said at least partially retracted eyelids when said portion of the eye is maintained vertically aligned under said distal aperture.

According to an embodiment of the present disclosure, said mechanism is an automatic electric mechanism.

According to an embodiment of the present disclosure, said mechanism is provided for receiving a squeezable eyedrop bottle and controllably delivering said predetermined quantity of liquid by squeezing said eyedrop bottle in a predetermined manner.

According to an embodiment of the present disclosure, said eyecup is arranged such that no part of the apparatus touches the eye directly.

According to an embodiment of the present disclosure, said opaque eye cup is made of a resilient material.

According to an embodiment of the present disclosure, said opaque eye cup is removably coupled to said mechanism.

According to an embodiment of the present disclosure, said eyecup distal aperture is arranged such that said portion of the eye exposed by said at least partially retracted eyelids is a predetermined portion of the eye where it is desired to drop said predetermined quantity of liquid.

According to an embodiment of the present disclosure, said mechanism is provided for controllably delivering said predetermined quantity of liquid upon actuation of a switch.

According to an embodiment of the present disclosure, said switch is arranged remotely from said mechanism.

According to an embodiment of the present disclosure, said opaque eye cup is arranged such that the user's eye cannot see said predetermined quantity of liquid as it drops into the eye.

According to an embodiment of the present disclosure, said apparatus comprises a support adapted to clamp to table or table leg and coupled to said mechanism; the support being arranged to maintaining said mechanism and said opaque eyecup at a predetermined position with respect to said table or table leg.

According to an embodiment of the present disclosure, said mechanism is electric.

According to an embodiment of the present disclosure, said mechanism comprises a rechargeable battery.

According to an embodiment of the present disclosure, said mechanism comprises a circuit arranged for keeping in memory how many times a same squeezable eye drop bottle was squeezed by the mechanism, and for adapting the strength of the squeezing of the bottle so that a same predetermined quantity of liquid is delivered each time the mechanism squeezes the bottle.

These and other features and advantages will become further apparent from the detailed description and accompanying figures that follow. In the figures and description, numerals indicate the various features, like numerals referring to like features throughout both the drawings and the description.

### BRIEF DESCRIPTION OF THE DRAWINGS

FIG. 1 illustrates schematically an apparatus for dispensing eye drops according to an embodiment of the present disclosure.

FIG. 2 illustrates schematically a top-front-right elevation view of an apparatus for dispensing eye drops according to an embodiment of the present disclosure.

FIG. 3 illustrates schematically a side-front-left elevation view of the apparatus of FIG. 1.

FIG. 4 illustrates schematically a front view of the apparatus of FIG. 1.

FIG. 5 illustrates schematically a front view of the apparatus of FIG. 1 with its front lid removed.

FIG. 6 illustrates schematically a back view of the apparatus of FIG. 1.

FIG. 7 illustrates schematically a right view of the apparatus of FIG. 1.

FIG. 8 illustrates schematically a squeezing mechanism transforming rotation into translation of the apparatus of FIG. 1.

FIG. 9 illustrates schematically how the motor and squeezing mechanism can be assembled in the apparatus of FIG. 1.

FIG. 10 illustrates schematically the apparatus of FIG. 1 held in the hand of a user, with its lid open and ready to receive a bottle of eye drop liquid.

FIG. 11 illustrates schematically an eye cup part of the apparatus of FIG. 1.

FIG. 12 illustrates schematically the lid part of the apparatus of FIG. 1.

FIG. 13 illustrates schematically the apparatus of FIG. 1 coupled to a support.

### DETAILED DESCRIPTION

In the following description, numerous specific details are set forth to clearly describe various specific embodiments disclosed herein. One skilled in the art, however, will understand that the presently claimed invention may be practiced without all of the specific details discussed below. In other instances, well known features have not been described so as not to obscure the invention. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present concept.

As detailed hereafter, an embodiment of the present disclosure comprises an eye drop bottle dispenser apparatus with a rubber cup at an end which matches with the shape of the user's eye. The cup allows the user to guide the device directly to the eye and to keep the user from closing their eye lids, to maximize the accuracy. The dispenser preferably contains a small cover in the front which when opened allows the user to place an eye drop bottle inside the device and with the help of a motor-actuated squeezing mechanism the device compresses the eye drop bottle from the bottom causing a single drop to fall directly into the user's eyeball. This apparatus allows elderly people to apply eye drops with dexterity without the help of another person. The automatic squeezing helps people with little strength in the hand to squeeze effortlessly eye drop bottles that otherwise would be too rigid for them to squeeze without shaking. The device optionally comes with a stand that helps disabled people to not use their hands to squeeze and hold the eye drop bottle. Pictorial representation of the eye dispenser is shown for better understanding the mechanism. However, it is evident to the person skilled in the art that the same function can be established by various design artifacts.

It is to be appreciated that the optimum dimensional relationships for the parts of an apparatus according to the present disclosure to include variations in size, materials, shape, form, function and manner of operation, assembly and use are deemed readily apparent and obvious to a person skilled in the art and all equivalent variations to the ones described in the specification are intended to be encompassed by the present invention.

Therefore, it needs to be appreciated that the aforementioned description is just an illustration of the principles of an apparatus according to an embodiment of the present disclosure. Since numerous modifications, variations, improvements and changes will readily occur to those skilled in the art, it is considered to limit the claimed apparatus to the exact described format of the claimed apparatus or its manufacturing and accordingly all suitable modifications and equivalents may be falling within the scope of the claims.

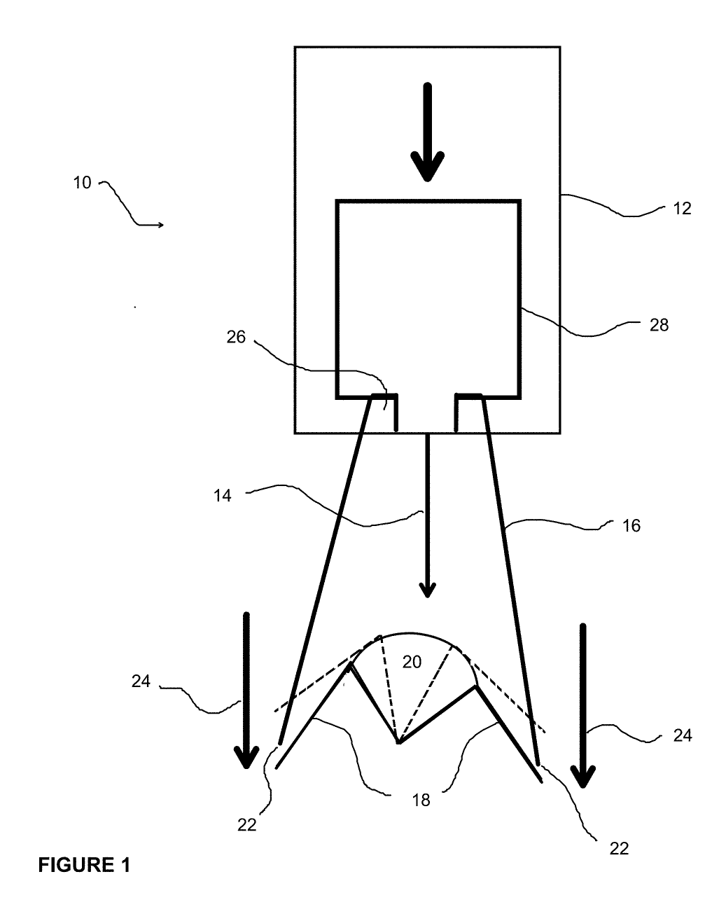

As illustrated in FIG. 1, an embodiment of the present disclosure comprises an apparatus 10 for dispensing eyedrops; the apparatus comprising: a mechanism in a housing 12 provided for controllably delivering a predetermined quantity of liquid 14; and an opaque eye cup 16 provided for at least partially retracting the eyelids 18 of a user's eye 20 when the opaque eyecup 16 is applied around the user's eye. According to an embodiment of the present disclosure, opaque eyecup 16 is shaped such that, when applied around a user's eye 20, a proximal end 22 of the eyecup 16 gently pushes 24 on the periphery of the eye 20 and pulls open the eyelids 18.

According to an embodiment of the present disclosure, eye cup 16 has a distal end 26 with an aperture and is coupled to mechanism 12 such that said predetermined quantity of liquid 14 drops through said distal aperture 26 into a portion of the eye 20 exposed by said at least partially retracted eyelids 18 when said portion of the 20 is maintained vertically aligned under said distal aperture. According to an embodiment of the present disclosure, eyecup 16 is made of an opaque material and is coupled to mechanism 12 such that only a limited quantity of light reaches the eye 20 when eyecup 16 pulls open the eyelids 18. Preferably, the eye is maintained sufficiently in the dark by the eyecup, so that the eye cannot see the eyedrop/predetermined quantity of liquid 14 form or fall toward the eye. This advantageously allows preventing the user from seeing the predetermined quantity of liquid 14 when it drops into the eye 20, thus preventing a reflex closure of the eye that in known eyedrop dispensers often cause a loss of the drop.

According to an embodiment of the present disclosure, mechanism 12 is an automatic electric mechanism; which is for example provided for receiving a squeezable eyedrop bottle 28 and for controllably delivering said predetermined quantity of liquid by squeezing eyedrop bottle 28 in a predetermined manner. Said predetermined quantity of liquid can be a drop of liquid of a desired volume; for example of between 0.07 ml and 0.03 ml cubic centimeter; preferably 0.05 ml cubic centimeter. As detailed hereafter, mechanism 12 can comprise a switchable electrical motor that actuates a squeezing mechanism which comprises at least a cam for transforming the rotational movement of the motor into a translational movement of a part provided for squeezing the eyedrop bottle. According to an embodiment of the present disclosure, the switch of the motor of mechanism 12 can be arranged remotely from mechanism 12, for example via a wire or wireless connection. According to an embodiment of the present disclosure, mechanism 12 can comprise a rechargeable battery or can comprise one or more battery slots for receiving removable batteries. According to an embodiment of the present disclosure, mechanism 12 can be entirely mechanical and comprise a windable spring to actuate the squeezing mechanism.

According to an embodiment of the present disclosure, eye cup 16 is formed of a soft resilient material, such as silicone rubber, and is arranged to press on the skin or eyelid 18 on the periphery of eye 20 such that no part of the apparatus 10 actually touches the eye 20 directly while opening the eyelid. For example, eyecup 16 can comprise a trunk of a cone having an apex angle comprised between 15 degree and 45 degree, preferably between 20 degree and 40 and preferably of 30 degree.

According to an embodiment of the present disclosure, eye cup 16 is removably coupled to mechanism 12, and is disposable.

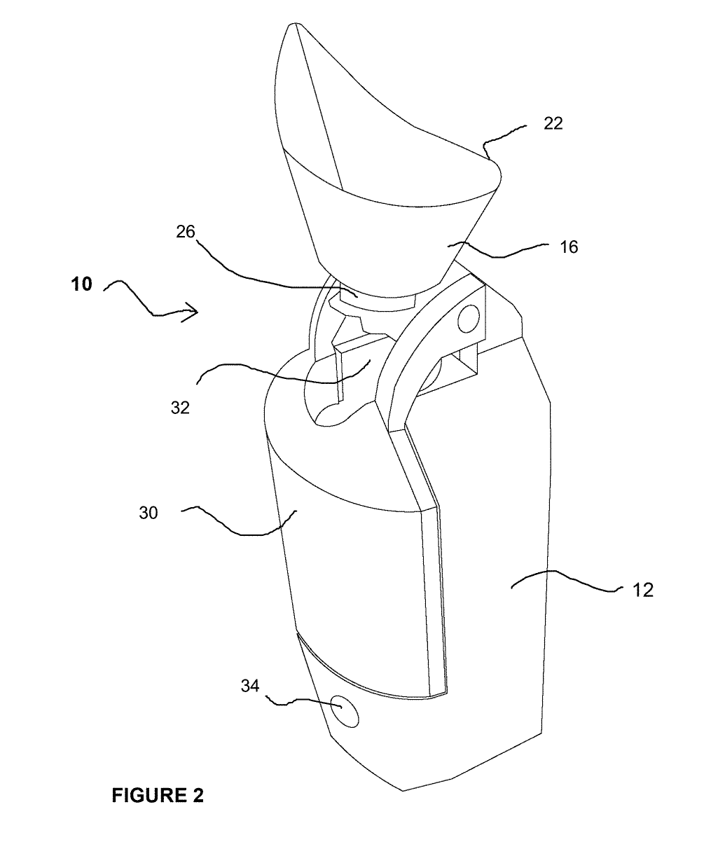

FIG. 2 illustrates schematically a top-front-right elevation view of an apparatus for dispensing eye drops according to an embodiment of the present disclosure, showing eye cup 16 attached at its distal end 26 to mechanism 12. According to an embodiment of the present disclosure, eye cup 16 can be asymmetric and in particular can have an asymmetric proximal end 22 with a short section to cup around the eye on the side of the nose and a long section to cup on the other side of the eye. The height of the short section of the trunk of a cone can be comprised between 1 centimeter and 6 centimeter; preferably between 2 and 5 centimeter, preferably of 3.5 centimeter. The height of the long section of the trunk of a cone can be comprised between 2.5 centimeter and 7.5 centimeter; preferably between 3.5 and 6.5 centimeter, and preferably of 5 centimeter.

According to an embodiment of the present disclosure, apparatus 10 can comprise a lid 30 hingedly connected to mechanism 12. Mechanism 12 can comprise a recess 32 arranged for receiving a squeezable eyedrop bottle when lid 30 is moved away from mechanism 12 to an “open” position (not illustrated in FIG. 2), and maintaining the eyedrop bottle in a position appropriate for being squeezed by the squeezing mechanism when lid 30 is moved toward mechanism 12 to an “closed” position (illustrated in FIG. 2). FIG. 2 also illustrates a switch 34 for actuating the squeezing mechanism.

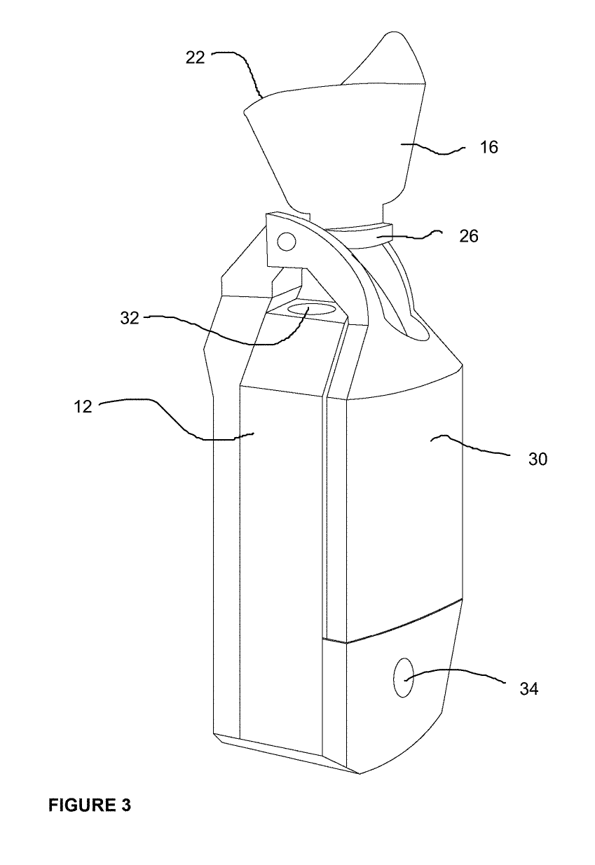

FIG. 3 illustrates schematically a side-front-left elevation view of the apparatus of FIG. 2. In the embodiment illustrated in FIG. 3, the recess 32 in mechanism 12 comprises a circular opening on the top of the housing of mechanism 12, through which the neck of a squeezable bottle (not shown) held in recess 32 can protrude toward the distal end 26 of eyecup 16.

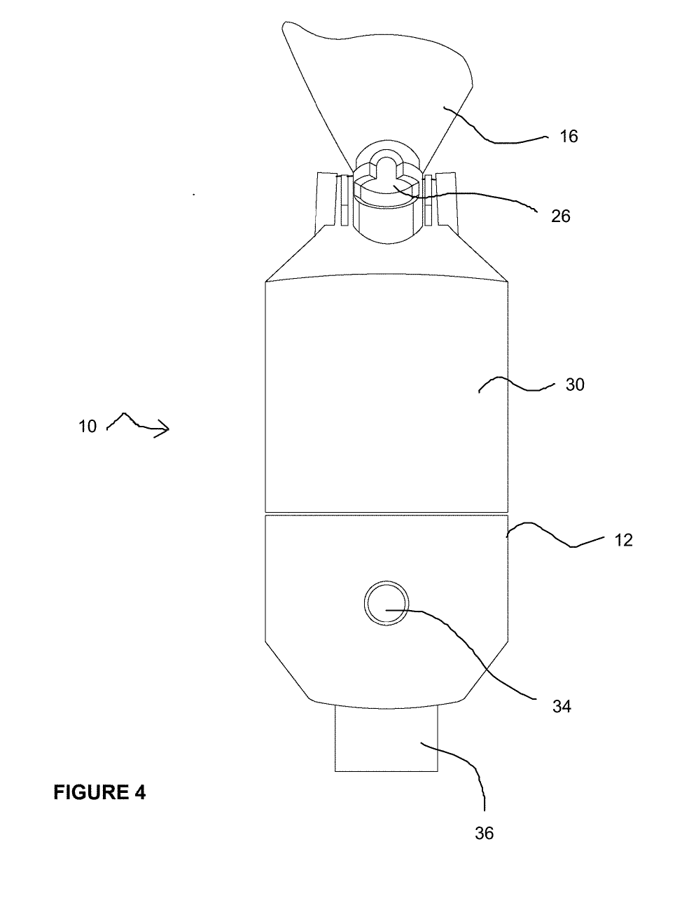

FIG. 4 illustrates schematically a front view of the apparatus of FIG. 1. According to an embodiment of this disclosure, the distal end 26 of eyecup 16 can be arranged to receive the neck of a bottle of eyedrop liquid in its opening. As illustrated in FIG. 4, the distal end 26 of eyecup 16 can then comprise a lateral slot that allows sliding the neck of the eyedrop bottle into the opening of the distal end 26 of eyecup 16. FIG. 4 further shows that the housing of mechanism 12 can comprise a coupling structure 36 for coupling apparatus 10 to a stand or support.

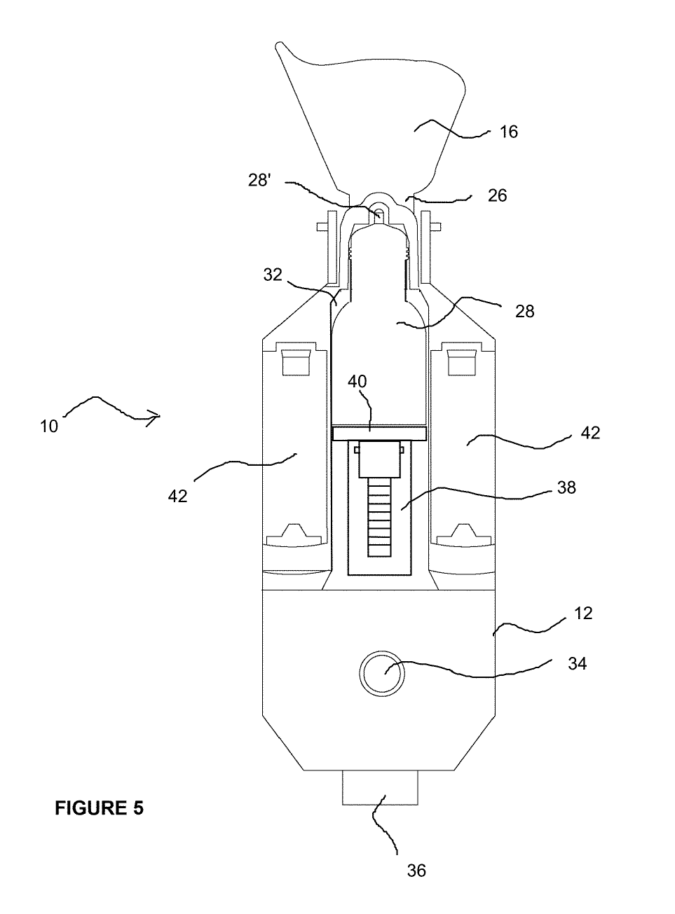

FIG. 5 illustrates schematically a front view of the apparatus 10 of FIG. 1 with its front lid removed, with an eyedrop bottle 28 arranged in recess 32; the neck 28′ of bottle 28 being arranged in the opening of distal end 26 of eyecup 16. According to an embodiment of the present disclosure, recess 32 can comprise a size-adjustable pushing structure 38 that allows adjusting the size of recess 32 to the type of bottle 28 that it is desired to be used in apparatus 10. Structure 38 can comprise a ratchet mechanism with a lock for controllably changing the position of a movable slab 40 that is arranged against the bottom of bottle 28. As detailed hereafter, slab 40 of pushing structure 38 is in an embodiment pushed against the end of bottle 28 by mechanism 12 upon actuation of switch 34. FIG. 5 also shows two exemplary battery recesses 42 for receiving batteries for powering the motor of mechanism 12.

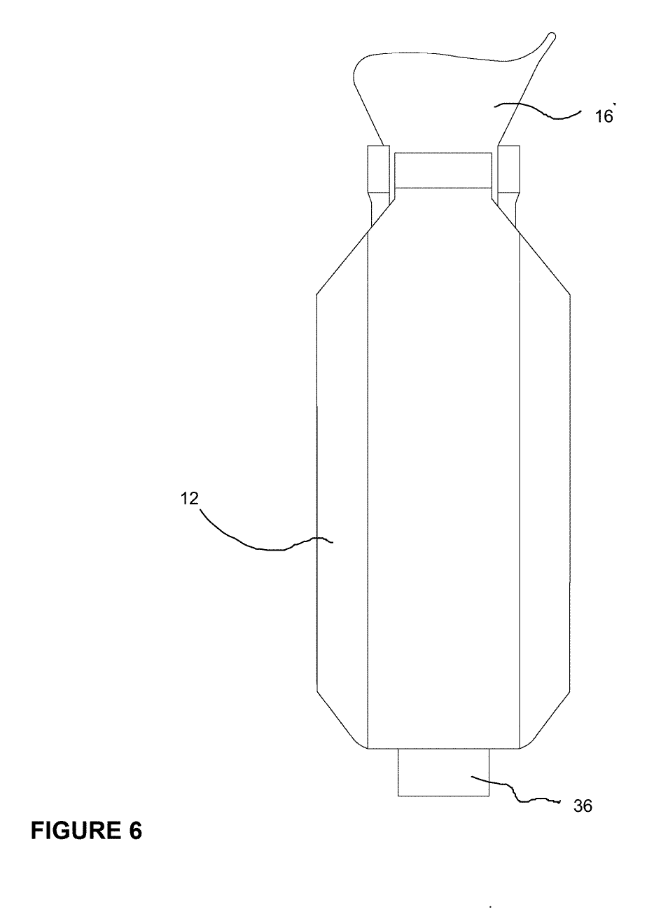

FIG. 6 illustrates schematically a back view of the apparatus of FIG. 1, with coupling structure 36, housing 12 and eyecup 16.

FIG. 7 illustrates schematically a right view of the apparatus of FIG. 1 with coupling structure 36, housing 12, lid 30, switch 34 and eyecup 16.

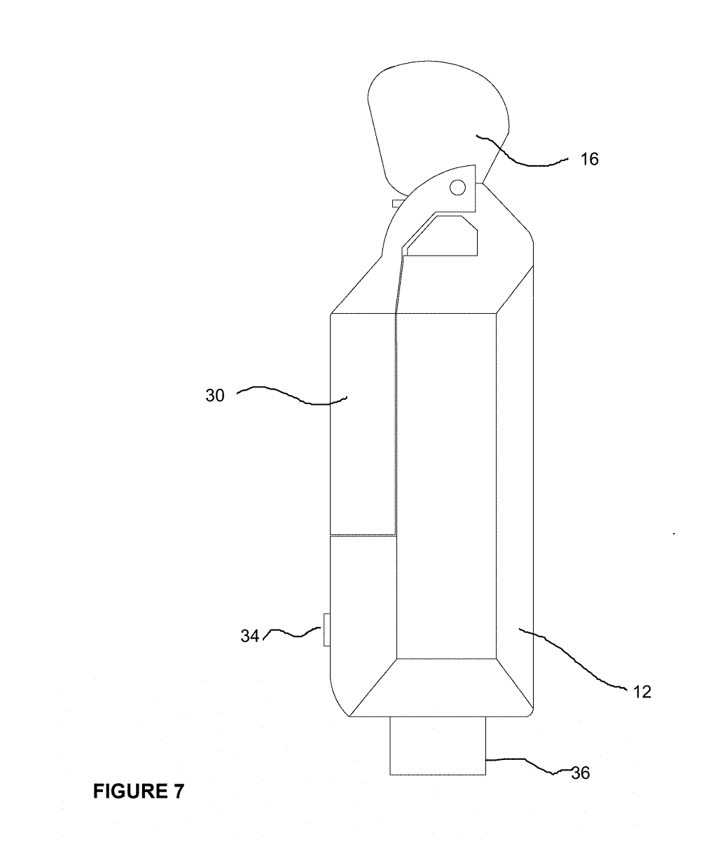

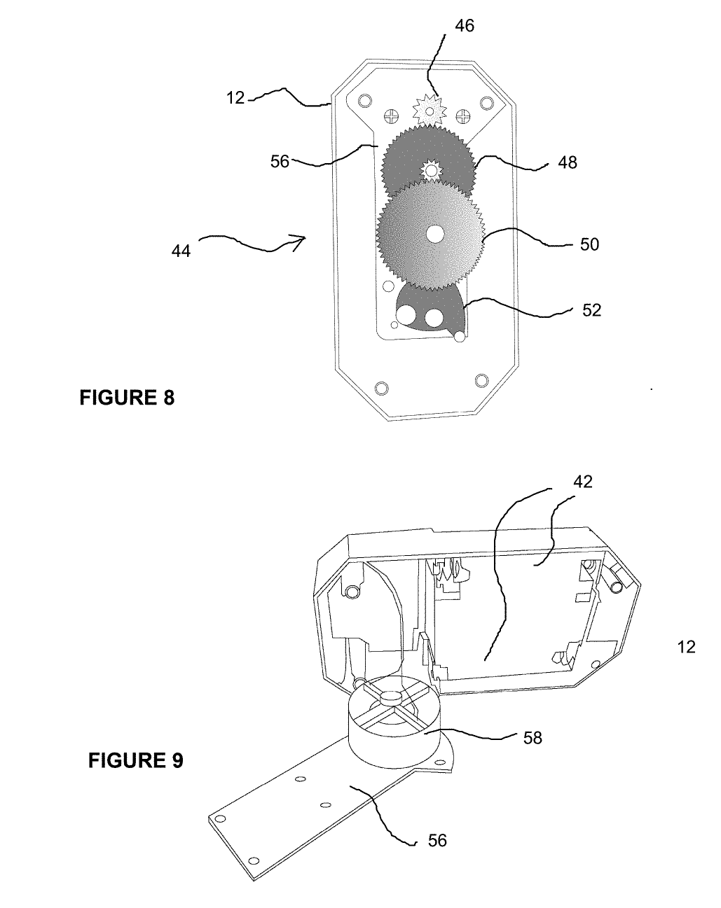

FIG. 8 illustrates schematically a mechanism 44 transforming rotation into translation in the housing of mechanism 12. According to an embodiment of the present disclosure, mechanism 44 comprises a motor output cogwheel 46 attached to the axis of an electric motor (not shown), two speed reduction cogwheels 48, 50 coupled to cogwheel 46 and a cam 52 that transforms the rotational motion of the slowest cogwheel 50 into a translation motion of the slab 40 (not shown; see FIG. 5) along the longitudinal axis of mechanism 44, so as to squeeze the eyedrop bottle 28 (not shown; see FIG. 5) when the motor is in rotation. According to the illustrated embodiments, the cogwheels 46, 48, 50 and cam 52 are arranges for rotating around axles normal to a front side of a plate 56 that is itself provided for being attached to the inside of the housing of the mechanism 12.

FIG. 9 illustrates an electric motor 58 attached to a back side of plate 56, with plate 56 removed from the inside of the housing of the mechanism 12, and flipped to expose the back side of plate 56. FIG. 9 also illustrates that the recesses 42 for receiving the batteries can simply be formed between a portion of the inside of the housing of the mechanism 12 and an edge of plate 56 when plate 56 is assembled into the inside of the housing of the mechanism 12. According to an embodiment of the present disclosure, motor 58 is actuated by switch 34. According to an embodiment of the present disclosure, switch 34 is arranged for, when pressed, actuating motor 58 for a predetermined time so as to squeeze bottle 28 in a predetermined manner and cause a drop of predetermined size of the liquid in bottle 28 to drop from the neck of bottle 28. Switch 34 can be coupled to an electronic circuit that keeps in memory how many times a single bottle was squeezed, so as to eventually adjust the strength that has to be exerted by mechanism 44 on bottle 28 to generate always a drop of a desired predetermined size. The memory of such a circuit can be initialized for example when the bottle 28 is removed. According to an embodiment of the present disclosure, mechanism 44, along with motor 58, and adjustment structure 38 form the bulk of mechanism 12.

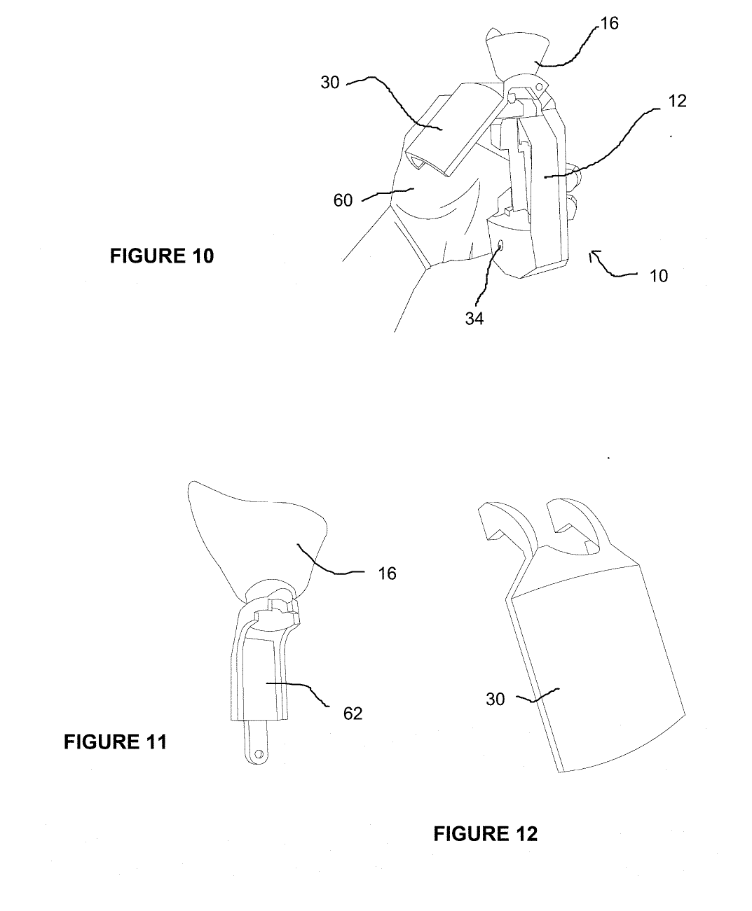

FIG. 10 illustrates schematically the apparatus 10 of FIG. 1 held in the hand 60 of a user, with its lid 30 open and ready to receive a bottle of eye drop liquid. FIG. 10 illustrates that a preferred size of apparatus is of between 10 and 15 centimeters.

FIG. 11 illustrates schematically an eye cup 16 of the apparatus 10 of FIG. 1 which would be placed over the users eye and used to guide the eye-drop correctly. According to an embodiment of the present disclosure, eyecup 16 can comprise a mounting flange 62 for ease of coupling to the housing of the mechanism 12.

FIG. 12 illustrates schematically the lid 30 of the apparatus 10 of FIG. 1. This lid covers the front part of the apparatus which protects the battery slots and the eye drop.

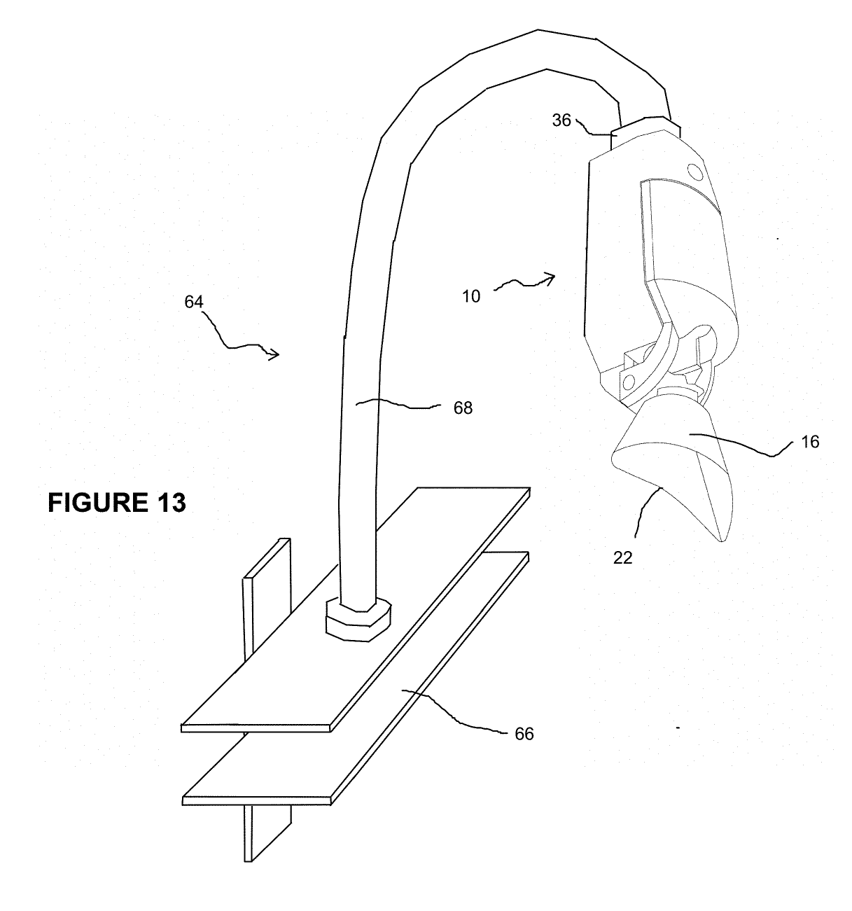

FIG. 13 illustrates schematically the apparatus 10 of FIG. 1 coupled to a support 64 capable of being attached to a surface or a support. According to an embodiment of the present disclosure, the support 64 comprises a clamp 66, arranged to firmly but removably grasp the edge or the foot of a table, and a flexible elongated member that can be bent into multiple shapes, having one end coupled to clamp 66 and one end removably couplable to coupling structure 36 of apparatus 10.

According to an embodiment of the present disclosure, support 64 is capable of maintaining mechanism 10, and consequently opaque eyecup 16, at a predetermined position, the eyecup 16 having its proximal end 22 facing downward.

Having now described the invention in accordance with the requirements of the patent statutes, those skilled in this art will understand how to make changes and modifications to the present invention to meet their specific requirements or conditions. Such changes and modifications may be made without departing from the scope and spirit of the invention as disclosed herein.

The foregoing Detailed Description of exemplary and preferred embodiments is presented for purposes of illustration and disclosure in accordance with the requirements of the law. It is not intended to be exhaustive nor to limit the invention to the precise form(s) described, but only to enable others skilled in the art to understand how the invention may be suited for a particular use or implementation. The possibility of modifications and variations will be apparent to practitioners skilled in the art.

No limitation is intended by the description of exemplary embodiments which may have included tolerances, feature dimensions, specific operating conditions, engineering specifications, or the like, and which may vary between implementations or with changes to the state of the art, and no limitation should be implied therefrom. Applicant has made this disclosure with respect to the current state of the art, but also contemplates advancements and that adaptations in the future may take into consideration of those advancements, namely in accordance with the then current state of the art. It is intended that the scope of the invention be defined by the Claims as written and equivalents as applicable. Reference to a claim element in the singular is not intended to mean “one and only one” unless explicitly so stated. Moreover, no element, component, nor method or process step in this disclosure is intended to be dedicated to the public regardless of whether the element, component, or step is explicitly recited in the Claims. No claim element herein is to be construed under the provisions of 35 U.S.C. Sec. 112, sixth paragraph, unless the element is expressly recited using the phrase “means for . . . ” and no method or process step herein is to be construed under those provisions unless the step, or steps, are expressly recited using the phrase “comprising the step(s) of . . . .”

What is claimed is:

1. An apparatus for dispensing eyedrops comprising:
a mechanism provided for controllably delivering a predetermined quantity of liquid; and
an opaque eye cup provided for at least partially retracting the eyelids of a user's eye when applied around said user's eye;
said eye cup having a distal aperture and being coupled to said mechanism such that said predetermined quantity of liquid drops through said distal aperture into a portion of the eye exposed by said at least partially retracted eyelids when said portion of the eye is maintained vertically aligned under said distal aperture.

2. The apparatus of claim 1, wherein said mechanism is an automatic electric mechanism.

3. The apparatus of claim 1, wherein said mechanism is provided for receiving a squeezable eyedrop bottle and controllably delivering said predetermined quantity of liquid by squeezing said eyedrop bottle in a predetermined manner.

4. The apparatus of claim 1, wherein said eyecup is arranged such that no part of the apparatus touches the eye directly.

5. The apparatus of claim 1, wherein said opaque eye cup is made of a resilient material.

6. The apparatus of claim 1, wherein said opaque eye cup is removably coupled to said mechanism.

7. The apparatus of claim 1, wherein said eyecup distal aperture is arranged such that said portion of the eye exposed by said at least partially retracted eyelids is a predetermined portion of the eye where it is desired to drop said predetermined quantity of liquid.

8. The apparatus of claim 1, wherein said mechanism is provided for controllably delivering said predetermined quantity of liquid upon actuation of a switch.

9. The apparatus of claim 1, wherein said switch is arranged remotely from said mechanism.

10. The apparatus of claim 1, wherein said opaque eye cup is arranged such that the user's eye cannot see said predetermined quantity of liquid as it drops into the eye.

11. The apparatus of claim 1, comprising a support adapted to clamp to table or table leg and coupled to said mechanism; the support being capable of maintaining said mechanism and said opaque eyecup at a predetermined position.

12. The apparatus of claim 1, wherein said mechanism is electric.

13. The apparatus of claim 12, wherein said mechanism comprises a rechargeable battery.

14. The apparatus of claim 12, wherein said mechanism comprises a circuit arranged for keeping in memory how many times a same squeezable eye drop bottle was squeezed by the mechanism, and for adapting the strength of the squeezing of the bottle so that a same predetermined quantity of liquid is delivered each time the mechanism squeezes the bottle.
